(19) United States
(12) Patent Application Publication
(10) Pub. No.: US 2010/0114231 A1
(43) Pub. Date: May 6, 2010

(54) METHODS AND SYSTEMS TO MONITOR ISCHEMIA

(75) Inventor: Mark W. Kroll, Crystal Bay, MN (US)

Correspondence Address: PACESETTER, INC., 15900 VALLEY VIEW COURT, SYLMAR, CA, 91392-9221, US

(73) Assignee: PACESETTER, INC., Sylmar, CA (US)

(21) Appl. No.: 12/265,151

(22) Filed: Nov. 5, 2008

(51) Int. Cl. A61N 1/365

(52) U.S. Cl. 607/25

(57) ABSTRACT

An implantable medical device includes leads, a segment monitoring module, an impedance detection module and an ischemia module. The leads include electrodes that are configured to be positioned within a heart and that are capable of sensing cardiac signals having a segment of interest. The segment monitoring module determines segment variations of the segment of interest in the cardiac signals. The impedance detection module measures impedance vectors between predetermined combinations of the electrodes. The ischemia detection module monitors ischemia based on changes in the segment variations of the segment of interest and based on changes in the impedance vectors.

### FIELD OF THE INVENTION

Embodiments of the present invention pertain generally to implantable and external medical devices and more particularly pertain to methods and systems that monitor ischemia.

### BACKGROUND OF THE INVENTION

An implantable medical device is implanted in a patient to monitor, among other things, electrical activity of a heart and to deliver appropriate electrical and/or drug therapy, as required. Implantable medical devices (“IMDs”) include, for example, pacemakers, cardioverters, defibrillators, implantable cardioverter defibrillators (“ICD”), and the like. The electrical therapy produced by an IMD may include, for example, pacing pulses, cardioverting pulses, and/or defibrillator pulses to reverse arrhythmias (e.g., tachycardias and bradycardias) or to stimulate the contraction of cardiac tissue (e.g., cardiac pacing) to return the heart to its normal sinus rhythm.

Cardiac ischemia is a condition whereby the heart tissue does not receive adequate amounts of oxygen and usually is caused by a blockage of an artery leading to the heart tissue. Ischemia arises during angina, coronary angioplasty, and any other condition that compromises blood flow to a region of tissue. When blockage of an artery is sufficiently severe, the cardiac ischemia becomes an acute myocardial infarction (“AMI”), which also is referred to as a myocardial infarction (“MI”) or a heart attack.

Many patients at risk of cardiac ischemia have pacemakers, ICDs or other medical devices implanted therein. Electrocardiograms (“ECG”) are useful for diagnosing ischemia and locating damaged areas within the heart. ECGs are composed of various waves and segments that represent the heart depolarizing and repolarizing. The ST segment in an ECG represents the portion of the cardiac signal between ventricular depolarization and ventricular repolarization. While P-waves, R-waves, and T-waves in the ECG may generally be considered features of a surface ECG, for convenience and generality, herein the terms R-wave, T-wave, and P-wave are also used to refer to the corresponding internal cardiac signal, such as an intra-cardiac electrogram (“IEGM”) signal. Techniques have been developed for detecting cardiac ischemia using implanted medical devices by identifying variations in the ST segment from the baseline cardiac signal that occur during cardiac ischemia. Deviation of the ST segment from a baseline is a result of injury to cardiac muscle, variations in the synchronization of ventricular muscle depolarization, drug or electrolyte influences, or the like. Some conventional techniques monitor the initiation of ischemia by determining a change in the ST segment. But not all ischemic events progress to the state of an AMI. One difference between ischemia and AMI is that ischemia generally is reversible without producing permanent cardiac tissue damage. Therefore, ischemia may occur but may not present itself as an AMI.

Conventional ischemia detection techniques have been proposed to detect and monitor ischemia. Conventional ischemia detection techniques primarily rely on identifying variations in the ST segment from the baseline cardiac signal that occur during cardiac ischemia. Yet, the ST segment is influenced by a large number of factors unrelated to ischemia. By way of example only, the ST segment may be influenced by the presence of drugs; electrolyte abnormalities; neurogenic factors such as a previous stroke, hemorrhage, tumor, and the like; and metabolic factors such as hypoglycemia and hyperventilation. Thus, relying solely on identifying variations in the ST segment to diagnose ischemia can be an unreliable manner of monitoring ischemia. An improved method and system are needed to detect and monitor ischemia.

### BRIEF SUMMARY OF THE INVENTION

In one embodiment, an implantable medical device is provided that includes leads, a segment monitoring module, an impedance detection module and an ischemia module. The leads include electrodes that are configured to be positioned within a heart and that are capable of sensing cardiac signals having a segment of interest. The segment monitoring module determines segment variations of the segment of interest in the cardiac signals. The impedance detection module measures impedance vectors between predetermined combinations of the electrodes. The ischemia detection module monitors ischemia based on changes in the segment variations of the segment of interest and based on changes in the impedance vectors.

In another embodiment, a method is provided for monitoring ischemia that includes providing leads having electrodes configured to be positioned within a heart and sensing, with the electrodes, cardiac signals having a segment of interest. Additionally, the method also includes determining segment variations of the segment of interest in the cardiac signals and measuring impedance vectors between predetermined combinations of the electrodes. The method includes monitoring ischemia based on changes in the segment variations of the segment of interest and based on changes in the impedance vectors.

In another embodiment, a computer readable storage medium for use in a medical device includes a memory and a programmable controller. The computer readable storage medium includes instructions to direct the memory to store cardiac signals sensed by electrodes positioned within a heart. The cardiac signals have a segment of interest. The instructions also direct the memory to store impedance vectors that are measured between predetermined combinations of the electrodes. The instructions direct the controller to determine segment variations of the segment of interest in the cardiac signals and to monitor ischemia based on changes in the segment variations of the segment of interest and based on changes in the impedance vectors.

### BRIEF DESCRIPTION OF THE DRAWINGS

In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

FIG. 1 illustrates an IMD that is coupled to a heart according to one embodiment.

FIG. 2 illustrates a single cardiac cycle that includes a P-wave, a Q-wave, an R-wave, an S-wave, and a T-wave.

FIG. 3 illustrates a block diagram of exemplary internal components of the IMD shown in FIG. 1.

FIG. 4 illustrates a process for monitoring ischemia in accordance with one embodiment.

FIG. 5 illustrates the ischemia-related parameters obtained at and ischemia indicators calculated at according to one embodiment.

FIG. 6 illustrates an alternative manner of measuring the third impedance vector Z₃.

FIG. 7 illustrates the third impedance vector Z₃ over a range of frequencies of the current I₃.

FIG. 8 illustrates a functional block diagram of an external device that is operated to interface with the IMD shown in FIG. 1 according to one embodiment.

FIG. 9 illustrates a distributed processing system in accordance with one embodiment.

FIG. 10 illustrates a block diagram of exemplary manners in which embodiments of the present invention may be stored, distributed and installed on a computer-readable medium.

### DETAILED DESCRIPTION

In the following detailed description, reference is made to the accompanying drawings which form a part hereof, and in which are shown by way of illustration specific embodiments in which the present invention may be practiced. These embodiments, which are also referred to herein as “examples,” are described in sufficient detail to enable those skilled in the art to practice the invention. It is to be understood that the embodiments may be combined or that other embodiments may be utilized, and that structural, logical, and electrical variations may be made without departing from the scope of the present invention. For example, embodiments may be used with a pacemaker, a cardioverter, a defibrillator, and the like. The following detailed description is, therefore, not to be taken in a limiting sense, and the scope of the present invention is defined by the appended claims and their equivalents. In this document, the terms “a” or “an” are used, as is common in patent documents, to include one or more than one. In this document, the term “or” is used to refer to a nonexclusive or, unless otherwise indicated.

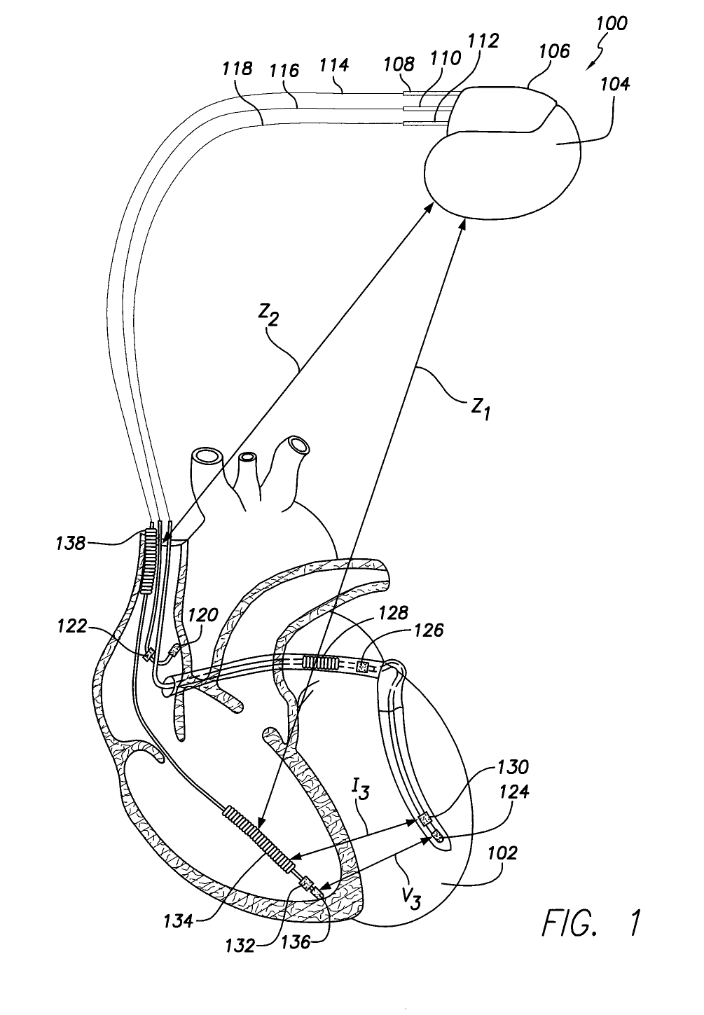

FIG. 1 illustrates an IMD 100 that is coupled to a heart 102. The IMD 100 may be a cardiac pacemaker, an ICD, a defibrillator, an ICD coupled with a pacemaker, and the like, implemented in accordance with one embodiment of the present invention. The IMD 100 may be a dual-chamber stimulation device capable of treating both fast and slow arrhythmias with stimulation therapy, including cardioversion, defibrillation, and pacing stimulation, as well as capable of detecting heart failure, evaluating its severity, tracking the progression thereof, and controlling the delivery of therapy and warnings in response thereto. As explained below in more detail, the IMD 100 may be controlled to monitor cardiac signals and based thereof, to identify potentially abnormal physiology (e.g. ischemia).

The IMD 100 includes a housing 104 that is joined to a header assembly 106 (e.g., an IS-4 connector assembly) that holds receptacle connectors 108, 110, 112 that are connected to a right ventricular lead 114, a right atrial lead 116, and a coronary sinus lead 118, respectively. The leads 114, 116, and 118 may be located at various locations, such as an atrium, a ventricle, or both to measure the physiological condition of the heart 102. One or more of the leads 114, 116, and 118 detect IEGM signals that form an electrical activity indicator of myocardial function over multiple cardiac cycles. To sense atrial cardiac signals and to provide right atrial chamber stimulation therapy, the right atrial lead 116 has at least an atrial tip electrode 120, which typically is implanted in the right atrial appendage, and an atrial ring electrode 122. The IEGM signals represent analog signals that are subsequently digitized and analyzed to identify waveforms or segments of interest. Examples of waveforms or segments of interest identified from the IEGM signals include the P-wave, T-wave, the R-wave, the QRS complex, the ST segment, and the like. The waveforms of interest may be collected over a period of time.

The coronary sinus lead 118 receives atrial and ventricular cardiac signals and delivers left ventricular pacing therapy using at least a left ventricular (“LV”) tip electrode 124, delivers left atrial (“LA”) pacing therapy using at least a left atrial ring electrode 126, and delivers shocking therapy using at least an LA coil electrode 128. The coronary sinus lead 118 also is connected with a LV ring electrode 130 disposed between the LV tip electrode 124 and the left atrial ring electrode 126. The LV ring electrode 130 may be used as a defibrillation electrode. The right ventricular (“RV”) lead 114 has an RV tip electrode 136, an RV ring electrode 132, an RV coil electrode 134, and an SVC coil electrode 138. The RV lead 114 is capable of receiving cardiac signals, and delivering stimulation in the form of pacing and shock therapy to the right ventricle. The RV coil electrode 134 may be used as a defibrillation electrode. For purposes of measuring impedance vectors (as described below), the housing 104 may be referred to as an electrode.

The electrodes 124-138 may have intrinsic impedances that vary among the electrodes 124-138. For example, the impedance of each electrode 124-138 may vary based on the type of electrode 124-138. The impedance of an electrode is related to the size of the electrode. Typically, the larger the size of the electrode, the lower the impedance of the electrode. Pacing electrodes such as the RV and LV tip electrodes 136, 124 tend to be smaller than defibrillating electrodes such as the RV coil electrode 134 and the LV ring electrode 130. As a result, the RV coil electrode 134 and the LV ring electrode 130 may have a lower impedance than the RV and/or LV tip electrodes 136, 124. For example, the LV and RV electrode tips 124 and 136 may have intrinsic impedances of at least 500 ohms while the RV coil electrode 134 and/or LV ring electrode 130 may have intrinsic impedances of approximately 100 ohms or less.

The IMD 100 monitors ischemia in the heart 102 by determining variations in impedance vectors and cardiac signals of the heart 102 between different sets of cardiac cycles. The IMD 100 measures and/or calculates several ischemia-related parameters to monitor and determine variations in the impedance measurements and cardiac signals. As described in more detail below, the IMD 100 may determine that the heart 102 is ischemic based on the number of differences and/or the extent of the differences among the cardiac signals and/or impedance vectors measured in different sets of cardiac cycles.

In the myocardium, healthy points or regions exhibit an impedance characteristic that is representative of the impedance of the tissue and the impedance of the blood at the point or in the regions. When the myocardium develops ischemia, regions of the myocardium that are ischemic exhibit a different impedance characteristic as compared to the same regions before becoming ischemic. Also, ischemic regions exhibit a different impedance characteristic as compared to surrounding healthy regions of the myocardium. The impedance characteristics of different regions can be measured to obtain impedance parameters.

The IMD 100 is configured to measure and compare impedance parameters for different sets of cardiac cycles to determine if the heart 102 is ischemic. An impedance parameter includes an impedance vector that represents the impedance measured along a path (generally a linear path) between at least two points. One or more impedance vectors measured by the IMD 100 may extend through the heart 102. The impedance vectors that extend through the heart 102 represent the impedance of the myocardium and the blood in the heart 102 along the paths of the impedance vectors. Impedance vectors along different paths that pass through the heart 102 may provide an indication of whether certain regions or points in the heart 102 are ischemic. For example, the IMD 100 may measure impedance vectors that traverse the heart 102 for multiple sets of cardiac cycles. The IMD 100 compares the averages of the impedance vectors for each cardiac cycle and compares the average impedance vectors. In a healthy, non-ischemic heart 102, the average impedance vectors over time may remain approximately the same over multiple sets of cardiac cycles.

For example, the myocardium of the non-ischemic heart 102 may have an intrinsic impedance of 50 ohms. In an ischemic heart 102, the impedance of the myocardium in the heart 102 may increase as the impedance vectors are measured by the IMD 100. For example, the intrinsic impedance of the myocardium of the heart 102 may increase by approximately 5 ohms or more. As a result, the average impedance vectors of a previous set of cardiac cycles may be less than the average impedance vectors of more recent set of cardiac cycles. If the difference between the average impedance vectors between the sets of cardiac cycles is larger than a predetermined threshold, the IMD 100 may determine that the heart 102 is ischemic.

By way of example only, the impedance vectors measured by the IMD 100 may include first, second and third impedance vectors Z₁, Z₂ and Z₃ (FIG. 1) that are measured using predetermined combinations of the housing 104 and the electrodes 124-138. The housing 104 may be referred to herein as an electrode and as one of the electrodes used to measure one or more of the impedance vectors Z₁, Z₂ and Z₃. As shown in FIG. 1, the first impedance vector Z₁ extends along a path between the RV coil electrode 134 and the housing 104 that primarily traverses the heart 102. The second impedance vector Z₂ extends along a path that primarily traverses a non-myocardial path between the SVC coil electrode 138 and the housing 104. For example, the second impedance vector Z₂ extends along a path that primarily traverses outside of the heart 102.

The IMD 100 may calculate the third impedance vector Z₃ using a four terminal measurement technique in one embodiment. The four terminal measurement technique may reduce the impact that the intrinsic impedance of the electrodes has on the third impedance vector Z₃. As described above, the intrinsic impedances of the electrodes 124-138 may be large when compared to the change in the impedance of the heart 102 caused by ischemia. For example, the LV and RV electrode tips 124, 136 may have intrinsic impedances of 500 ohms or more while the change in impedance of the myocardium in the heart 102 caused by ischemia may be approximately 5 ohms or less. The four terminal measurement technique can eliminate the intrinsic impedances of the LV and RV electrode tips 124, 136 from the measured third impedance vector Z₃.

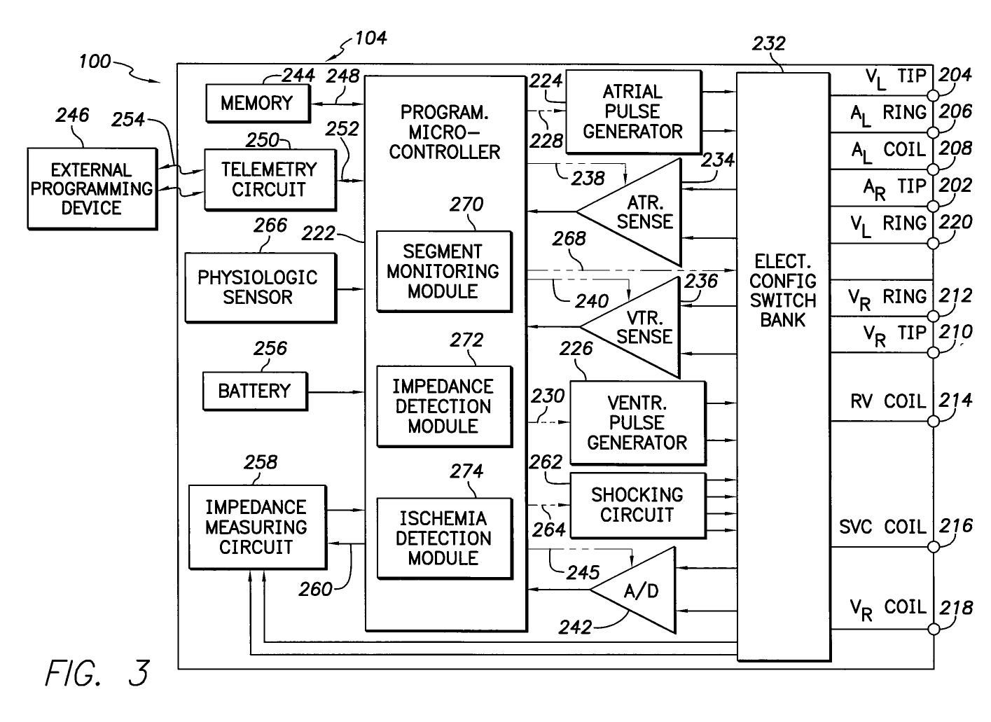

The four terminal measurement technique involves applying a current I₃ across a predetermined combination of the electrodes 124-136 while measuring a voltage V₃ between a different combination of the electrodes 124-136. As shown in FIG. 1, the current I₃ may be supplied between the RV coil electrode 134 and the LV ring electrode 130. The current I₃ can be supplied by electrically connecting the RV coil electrode 134 and the LV ring electrode 130 to a source of electric current, such as a battery 256 (shown in FIG. 3). The amount of current I₃ may controlled by an impedance detection module 272 (shown in FIG. 3). The voltage V₃ is measured between the LV tip electrode 124 and the RV tip electrode 136. The voltage V₃ represents the voltage difference measured between the LV tip electrode 124 and the RV tip electrode 136 when the current I₃ is supplied between the LV ring electrode 130 and the RV coil electrode 134. Using the voltage V₃ and the current I₃, the third impedance vector Z₃ may be calculated using the relationship:

$${Z}_{3} = \frac{{V}_{3}}{{I}_{3}} \qquad ( Eqn . 1 )$$

In one example, the IMD 100 measures one or more of the three impedance vectors Z₁, Z₂ and Z₃ for each cardiac cycle in a first set of cardiac cycles. By way of example only, the set may include 10 cardiac cycles. The IMD 100 calculates an average value of one or more of the impedance vectors Z₁, Z₂ and Z₃ for the first set of cardiac cycles. The IMD 100 then measures one or more of the three impedance vectors Z₁, Z₂ and Z₃ for each cardiac cycle in a second set of cardiac cycles. The IMD 100 calculates an average value of one or more of the impedance vectors Z₁, Z₂ and Z₃ for the second set of cardiac cycles. The average values of one or more of the impedance vectors Z₁, Z₂ and Z₃ between the sets of cardiac cycles are then compared. If the difference between the averages of the impedance vectors Z₁, Z₂ and Z₃ is great enough, the IMD 100 may determine that the heart 102 is ischemic.

One or more of the impedance vectors Z₁, Z₂ and Z₃ may be impacted by physiological conditions unrelated to ischemia. For example, the measured value of one or more of the impedance vectors Z₁, Z₂ and Z₃ may be different from the actual value of the impedance vectors Z₁, Z₂ and Z₃ if the patient being monitored breathes or has fluid in his/her lungs. The patient's breathing or fluid in the lungs can cause one or more of the impedance vectors Z₁, Z₂ and Z₃ to be measured as a different value than would otherwise be measured if the patient were holding his/her breath or did not have fluid in his/her lungs.

With respect to the variations in the cardiac signals, the IMD 100 can measure and identify ischemia-related parameters from cardiac signals of the heart 102. The ischemia-related parameters can include one or more segments of interest and/or variations in the segments of interest. For example, the IMD 100 can measure the R-wave in a QRS complex, the ST segment that follows the QRS complex, and variations in the R-wave and/or ST segment in multiple sets of cardiac cycles.

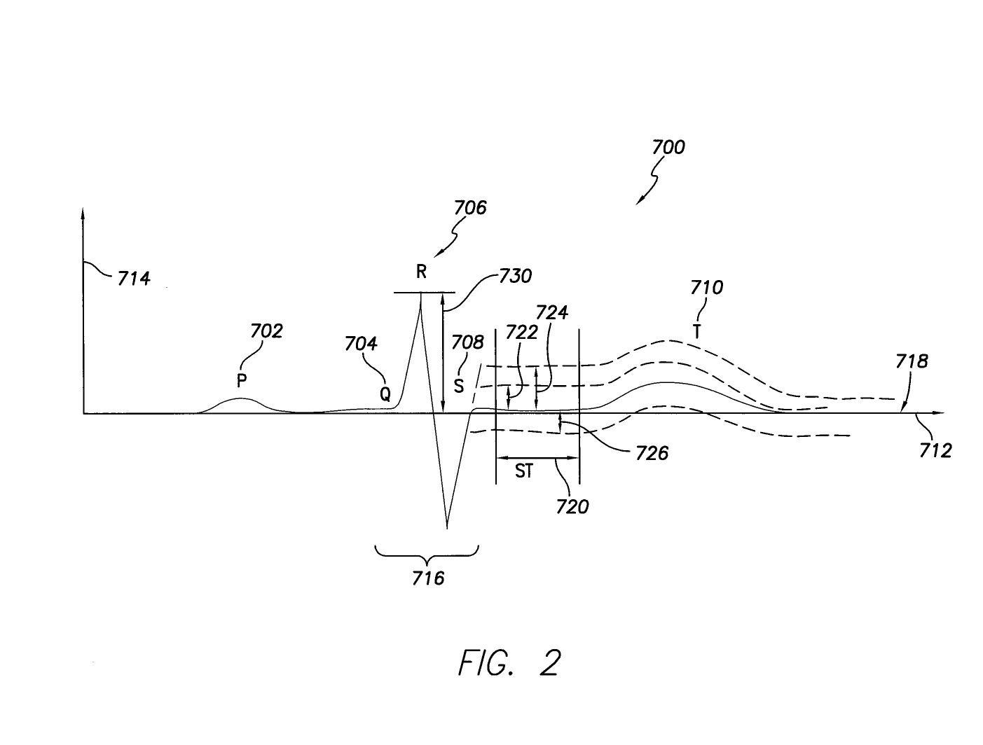

FIG. 2 illustrates a single cardiac cycle 700 that includes a P-wave 702, a Q-wave 704, an R-wave 706, an S-wave 708, and a T-wave 710. The cardiac cycle 700 may represent cardiac signals, such as IEGM signals, ECG signals, and the like. The horizontal axis 712 represents time, while the vertical axis 714 is defined in units of voltage. A QRS complex 716 is composed of the Q-wave 704, the R-wave 706, and the S-wave 708. The QRS complex 716 is used to locate the R-wave 706 to determine a baseline 718. The portion of the signal between the S-wave 708 and T-wave 710 constitutes an ST segment 720.

In a non-ischemic heart 102, the R-wave 706 and the ST segment 720 remain approximately the same for a plurality of cardiac cycles and/or a plurality of sets of cardiac cycles. For example, an amplitude 730 of the R-wave 706 may be approximately the same for each R-wave 706 in a plurality of cardiac cycles in a set, and approximately the same for the cardiac cycles in a plurality of sets of cardiac cycles. In another example, the ST segment 720 may be located at approximately the same location with respect to a baseline 718 for each cardiac cycle in a set of cardiac cycles, and approximately the same for the cardiac cycles in a plurality of sets of cardiac cycles.

In an ischemic heart 102, however, the R-wave 706 and/or the ST segment 720 may differ between cardiac cycles or between sets of cardiac cycles. For example, the amplitude 730 of the R-wave 706 may increase or decrease between cardiac cycles or sets of cardiac cycles. In another example, the ST segment 720 may shift above 722, 724 or below 726 the baseline 718. The ST segment variations 722-726 may occur above or below the baseline 718 for an ischemic heart 102. For example, the ST segment variations 722-726 may arise because of differences in the electrical potential between cells that have become ischemic and those that are still receiving normal blood flow. Thus, the ST segment variations 722-726 may be some indicators of the possibility of ischemia. In one example, the IMD 100 may determine that the heart 102 is not ischemic if the R-wave 706 and/or ST 720 segment do not significantly change between multiple sets of cardiac cycles. Conversely, the IMD 100 may determine that the heart 102 is ischemic if the R-wave 706 and/or ST 720 segment do significantly change between the sets of cardiac cycles.

FIG. 3 illustrates a block diagram of exemplary internal components of the IMD 100. The IMD 100 is for illustration purposes only, and it is understood that the circuitry could be duplicated, eliminated or disabled in any desired combination to provide a device capable of treating the appropriate chamber(s) of the heart with cardioversion, defibrillation and/or pacing stimulation. The housing 104 for IMD 100 (shown schematically in FIG. 2), is often referred to as the “can”, “case” or “case electrode” and may be programmably selected to act as the return electrode for all “unipolar” modes. The housing 104 further includes a connector (not shown) having a plurality of terminals, namely a right atrial tip terminal (A_{R} TIP) 202, a left ventricular tip terminal (V_{L} TIP) 204, a left atrial ring terminal (A_{L} RING) 206, a left atrial shocking terminal (A_{L} COIL) 208, a right ventricular tip terminal (V_{R} TIP) 210, a right ventricular ring terminal (V_{R} RING) 212, a right ventricular shocking terminal (RV COIL) 214, an SVC shocking terminal (SVC COIL) 216, a right ventricular coil terminal (V_{R} COIL) 218 and a left ventricular ring terminal (V_{L} RING) 220.

The IMD 100 includes a programmable microcontroller 222, which controls the operation of the IMD 100 based on acquired cardiac signals and impedance vectors. The microcontroller 222 (also referred to herein as a processor module or unit) typically includes a microprocessor, or equivalent control circuitry, is designed specifically for controlling the delivery of stimulation therapy and may further include RAM or ROM memory, logic and timing circuitry, state machine circuitry, and I/O circuitry. Typically, the microcontroller 222 includes the ability to process or monitor input signals (e.g., data) as controlled by a program code stored in a memory. Among other things, the microcontroller 222 receives, processes, and manages storage of digitized data from the various electrodes 104, 124-138 (shown in FIG. 1). The microcontroller 222 may also analyze the data, for example, in connection with collecting, over a period of time, variations in a segment of interest and impedance vectors. For example, the microcontroller 222 monitors variations in one or more of segments of interest such as the ST segment 720 (shown in FIG. 2) and the R-wave 706 (shown in FIG. 2) and variations in impedance vectors between predetermined electrodes 104 and 124 through 138 to monitor and determine a potential ischemic condition.

The modules in the microcontroller 222 that monitor ischemia include a segment monitoring module 270, the impedance detection module 272 and an ischemia detection module 274. The segment monitoring module 270 determines segment variations such as ST segment variations 722-726 (shown in FIG. 2) and changes in the amplitude 730 (shown in FIG. 2) of the R-wave 706 (shown in FIG. 2). The impedance detection module 272 measures and/or calculates one or more of the first, second and third impedance vectors Z₁, Z₂ and Z₃ (shown in FIG. 1). The ischemia detection module 274 monitors a potential ischemic condition based on changes in the segment variations monitored by the segment monitoring module 270 and based on changes in the impedance vectors monitored by the impedance detection module 272.

The IMD 100 includes an atrial pulse generator 224 and a ventricular/impedance pulse generator 226 to generate pacing stimulation pulses. In order to provide stimulation therapy in each of the four chambers of the heart 102 (shown in FIG. 1), the atrial and ventricular pulse generators 224 and 226, may include dedicated, independent pulse generators, multiplexed pulse generators, or shared pulse generators. The pulse generators, 224 and 226, are controlled by the microcontroller 222 via appropriate control signals, 228 and 230, respectively, to trigger or inhibit the stimulation pulses.

Switch 232 includes a plurality of switches for connecting the desired electrodes, including the electrodes 104 and 124 through 138 (shown in FIG. 1), to the appropriate I/O circuits, thereby providing complete electrode programmability. The switch 232, in response to a control signal 268 from the microcontroller 222, determines the polarity of stimulation pulses (e.g., unipolar, bipolar, etc.) by selectively closing the appropriate combination of switches (not shown) as is known in the art. Atrial sensing circuits 234 and ventricular sensing circuits 236 may also be selectively coupled to the leads 114, 116 and 118 (shown in FIG. 1) through the switch 232 for detecting the presence of cardiac activity in each of the four chambers of the heart 102 (shown in FIG. 1). Control signals 238 and 240 from microcontroller 222 direct output of the atrial and ventricular sensing circuits 234 and 236 that are connected to the microcontroller 222. In this manner, the atrial and ventricular sensing circuits 234 and 236 are able to trigger or inhibit the atrial and ventricular pulse generators 224 and 226.

The cardiac signals are applied to the inputs of an analog-to-digital (A/D) data acquisition system 242. The data acquisition system 242 is configured to acquire IEGM signals, convert the raw analog data into a digital IEGM signals, and store the digital IEGM signals in a memory 244 for later processing and/or telemetric transmission to an external device 246.

A control signal 245 from the microcontroller 222 determines when the A/D 242 acquires signals, stores them in memory 244, or transmits data to the external device 246. The A/D 242 is coupled to the right atrial lead 116 (shown in FIG. 1), the coronary sinus lead 118 (shown in FIG. 1), and the right ventricular lead 114 through the switch 232 to sample cardiac signals across any combination of desired electrodes 124-138 (shown in FIG. 1). The microcontroller 222 is coupled to the memory 244 by a suitable data/address bus 248, wherein the programmable operating parameters used by the microcontroller 222 are stored and modified, as required, in order to customize the operation of IMD 100 to suit the needs of a particular patient. The memory 244 may also store data indicative of myocardial function, such as the IEGM data, ST segment shifts, reference ST segment shifts, ST segment shift thresholds, R wave amplitudes, R wave amplitude changes, impedance vectors, trend information associated with ischemic episodes, and the like for a desired period of time (e.g., 6 hours, 12 hours, 18 hours or 24 hours, and the like).

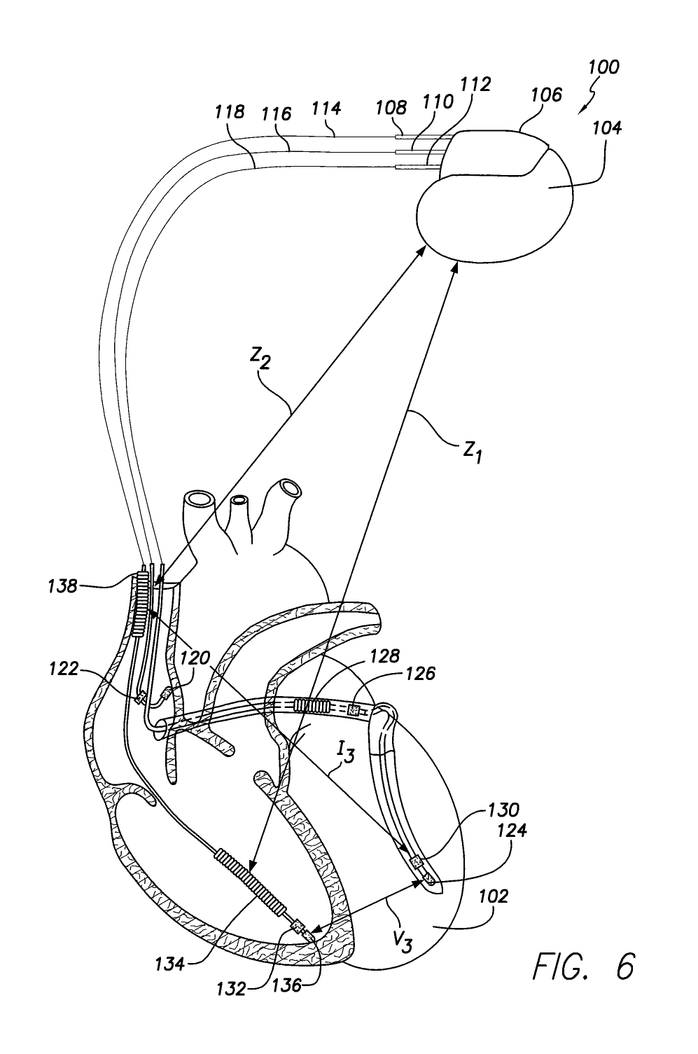

The operating parameters of the IMD 100 may be non-invasively programmed into the memory 244 through a telemetry circuit 250 in communication with the external device 246, such as an external device 400 (shown in FIG. 6), a trans-telephonic transceiver or a diagnostic system analyzer. The telemetry circuit 250 is activated by the microcontroller 222 by a control signal 252. The telemetry circuit 250 allows intra-cardiac electrograms and status information relating to the operation of IMD 100 (as contained in the microcontroller 222 or memory 244), to be sent to the external device 246 through an established communication link 254. The IMD 100 additionally includes the battery 256, which provides operating power to all of the circuits shown within the housing 104, including the microcontroller 222. The IMD 100 also includes a physiologic sensor 266 that may be used to adjust pacing stimulation rate according to the exercise state of the patient.

In the case where IMD 100 is intended to operate as an ICD device, the IMD 100 detects the occurrence of an ST segment shift 722-726 (shown in FIG. 2) that indicates an arrhythmia, and automatically applies an appropriate electrical shock therapy to the heart aimed at terminating the detected arrhythmia. To this end, the microcontroller 222 further controls a shocking circuit 262 by way of a control signal 264. The shocking circuit 262 generates shocking pulses of low (up to 0.5 joules), moderate (0.5-10 joules) or high energy (11 to 40 joules). Such shocking pulses are applied to the heart 102 (shown in FIG. 1) of the patient through at least two shocking electrodes, and as shown in this embodiment, selected from the left atrial coil electrode 128 (shown in FIG. 1), the RV coil electrode 134 (shown in FIG. 1), and/or the SVC coil electrode 138 (shown in FIG. 1).

The IMD 100 includes an impedance measuring circuit 258 which is enabled by the microcontroller 222 via a control signal 260. Alternatively, the impedance measuring circuit 258 is included in the impedance detection module 272. The impedance measuring circuit 258 is advantageously coupled to the switch 232 so that impedance at any desired electrode may be obtained. For example, the impedance measuring circuit 258 may measure impedance vectors between predetermined combinations of the electrodes 104 and 124 through 138 (shown in FIG. 1) to monitor and determine a potential ischemic condition.

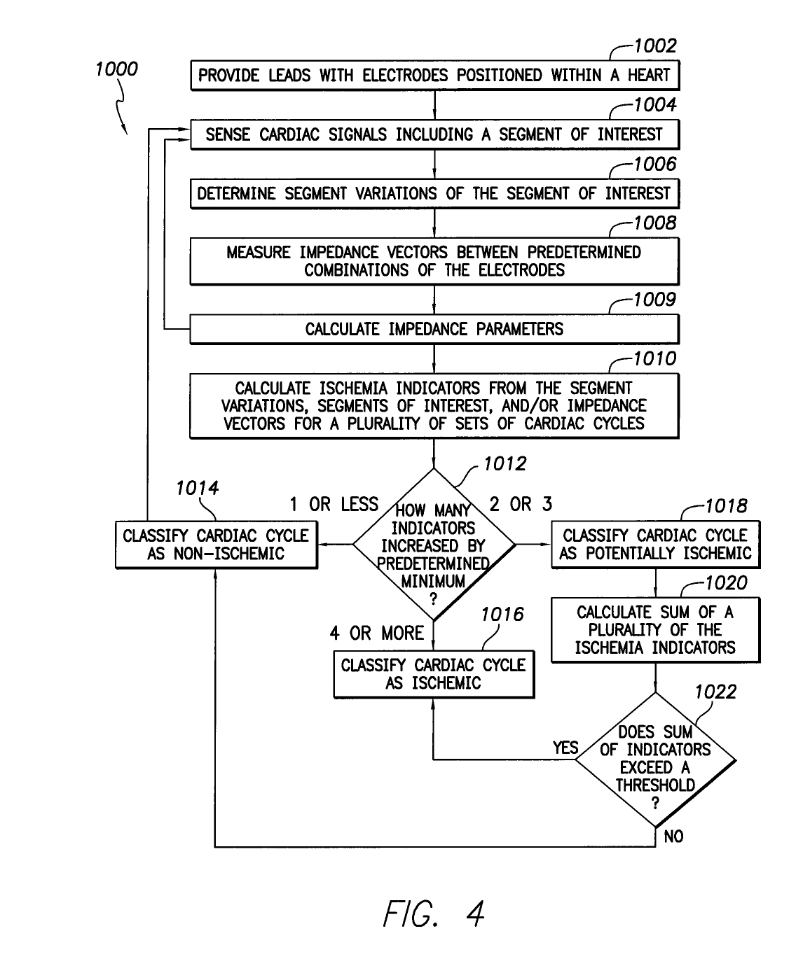

FIG. 4 illustrates a process 1000 for monitoring ischemia in accordance with one embodiment. At 1002, a plurality of leads 114, 116, and 118 (shown in FIG. 1) with electrodes 104 and 120-138 (shown in FIG. 1) is provided. The electrodes 120-138 are positioned within a heart 102 (shown in FIG. 1). As described above, the electrodes 104 and 120-138 can include pacing electrodes and defibrillation electrodes.

The IMD 100 determines several ischemia-related parameters at 1004-1009 for cardiac cycles in a set of cardiac cycles. For example, the segment monitoring module 270 may sense cardiac signals of the heart 102 at least some of the electrodes 104 and 120-138 at 1004. The cardiac signals are used to identify segments of interest, including one or more of the ST segment 720 (shown in FIG. 2) and the R-wave 706 (shown in FIG. 2), as described above. The IMD 100 (shown in FIG. 1) determines variations in one or more of the segments of interest at 1006. For example, the segment monitoring module 270 (shown in FIG. 3) may determine one or more variations 722-726 in the ST segment 720. In one example, the segment monitoring module 270 may determine the amplitude 730 of the R-wave 706 at 1006. The IMD 100 also measures and calculates the impedance vectors Z₁, Z₂ and Z₃ at 1008. For example, the impedance detection module 274 (shown in FIG. 3) and/or the impedance measuring circuit 258 (shown in FIG. 3) measure the first and second impedance vectors Z₁ and Z₂ and calculate the third impedance vector Z₃, as described above.

The IMD 100 calculates additional impedance parameters at 1009. For example, the impedance detection module 272 may calculate one or more additional impedance parameters. The additional impedance parameters 1009 may be calculated for each cardiac cycle in a set of cardiac cycles. The additional impedance parameters 1009 include an impedance normalization parameter Z_{N}. The normalized impedance parameter Z_{N} can be used to at least partially correct for variations in or more of the impedance vectors Z₁, Z₂ and Z₃ that are due to physiologic characteristics unrelated to ischemia. As described above, the measured value of one or more of the impedance vectors Z₁, Z₂ and Z₃ may be different from the actual value of the impedance vectors Z₁, Z₂ and Z₃ if the patient being monitored breathes during measurement of the vectors or has fluid in his/her lungs. The normalized impedance parameter Z_{N} may be defined by the following relationship:

$${Z}_{N} = \frac{{Z}_{1}}{{Z}_{2}} \qquad ( Eqn . 2 )$$

where Z₁ is the first impedance vector and Z₂ is the second impedance vector.

The impedance detection module 272 may calculate a contractility parameter C₃ and a normalized contractility parameter C_{N} at 1009. The contractility parameters C₃, C_{N} represent quantifiable parameters of the ability of the heart 102 to contract. Significant changes in one or more of the contractility parameters C₃, C_{N} between sets of cardiac cycles may indicate that the heart 102 is ischemic. The contractility parameter C₃ represents the rate of change in the third impedance vector Z₃ with respect to time. The contractility parameter C₃ may be represented by the following relationship:

$${C}_{3} = max \vert \frac{d {Z}_{3}}{d t} \vert \qquad ( Eqn . 3 )$$

For example, the contractility parameter C₃ may be the maximum value of the absolute value of the rate of change in the third impedance vector Z₃ with respect to time during a single cardiac cycle.

The normalized contractility parameter C_{N} represents the rate of change in the normalized impedance parameter Z_{N} with respect to time. The normalized contractility parameter C_{N} may be represented by the following relationship:

$${C}_{N} = max \vert \frac{d {Z}_{N}}{d t} \vert \qquad ( Eqn . 4 )$$

For example, the normalized contractility parameter C_{N} may be the maximum value of the absolute value of the change in the normalized impedance parameter Z_{N} with respect to time during a single cardiac cycle.

The segment monitoring module 270 may calculate a segment of interest (“SOI”) parameter at 1009. The SOI parameter represents a factor indicative of segment variations 722-726 (shown in FIG. 2) in the ST segment 720 (shown in FIG. 2) compared to the amplitude 730 (shown in FIG. 2) of the R-wave 706 (shown in FIG. 2). For example, if the segment variation 722-726 is small when compared to the amplitude 730, then the SOI parameter may have a small numerical value. Conversely, if the segment variation 722-726 approaches or exceeds the amplitude 730, then the SOI parameter may have a numerical value that approaches or exceeds 1. In one embodiment, the SOI parameter is the absolute value of the ratio of the ST segment parameter ST to the R-wave parameter R_{W}. For example, the SOI parameter may be defined by the following relationship:

$$S O I = \vert \frac{ST}{{R}_{w}} \vert \qquad ( Eqn . 5 )$$

where the ST segment parameter ST constitutes the segment variation 722-726 and the R-wave parameter R_{W} constitutes the amplitude 730 for a cardiac cycle.

In one embodiment, the IMD 100 determines the parameters at 1004-1009 for each cardiac cycle in a set of cardiac cycles. The IMD 100 performs the actions described at 1004-1009 for each cardiac cycle in the set until the cardiac signals, segment variations and impedance vectors have been determined for all of the cardiac cycles in the set. As shown in FIG. 4, the IMD 100 repeats the actions at 1004-1009 in a loop-wise manner until the parameters Z₁, Z₂, Z₃, Z_{N}, ST, R_{W}, C₃, C_{N} and SOI have been determined for all of the cardiac cycles in the set. The IMD 100 repeats the actions at 1004-1009 in a loop-wise manner for each cardiac cycle in another set of cardiac cycles.

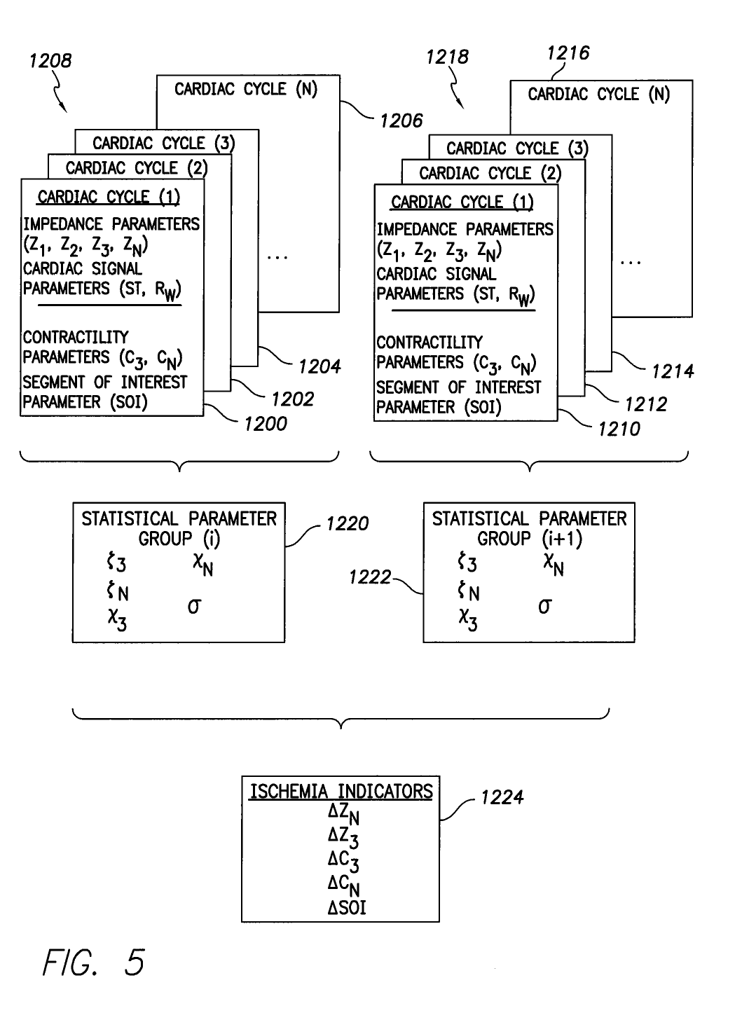

With continued reference to FIG. 4, FIG. 5 is a schematic illustration of the ischemia-related parameters obtained at 1004-1009 and ischemia indicators calculated at 1010 according to one embodiment. As shown in FIG. 5, the IMD 100 obtains several ischemia-related parameters for each cardiac cycle 1200-1206 in a first set 1208 of cardiac cycles and for each cardiac cycle 1210-1216 in a second set 1218 of cardiac cycles. For example, the IMD 100 may determine each of the impedance parameters Z₁, Z₂, Z₃, and Z_{N}, the cardiac signal parameters ST and R_{W}, the contractility parameters C₃ and C_{N} and the segment of interest parameter SOI for each of the cardiac cycles 1210-1216 in the first set 1208 and for each cardiac cycle 1210-1216 in the second set 1210.

The IMD 100 calculates ischemia indicators at 1010. The ischemia indicators may be calculated by the ischemia detection module 274. The ischemia indicators are representations of the degree or amount of change in the ischemia-related parameters between different sets of cardiac cycles. For example, the ischemia indicators represent how much the various ischemia-related parameters change between sets of cardiac cycles. A large change in the ischemia indicators between sets of cardiac cycles may indicate an ischemic or a potentially ischemic condition. For example, if a minimum number of the ischemia indicators exceed a minimum threshold, then the ischemia detection module 274 determines that the heart 102 is ischemic or potentially ischemic. By way of non-limiting example only, the ischemic detection module 274 determines how many of the ischemic indicators are at least 3%, or 0.03. Other thresholds such as 1%, 5%, 8%, 10%, and the like, may be used in place of the 3% individual threshold. The individual threshold may be stored in the memory 244 (shown in FIG. 3).

In one embodiment, the ischemia detection module 274 calculates the ischemia indicators by calculating a group 1220, 1222 of statistical parameters for each set 1208, 1218 of cardiac cycles. For example, the ischemia detection module 274 may calculate several statistical parameters for a first group 1220 and for a second group 1222. The statistical parameters are functions of one or more of the ischemia-related parameters for each set 1208, 1218 of cardiac cycles. For example, the ischemia detection module 274 may calculate a statistical impedance parameter ζ₃ for each set 1208, 1218 of cardiac cycles. The statistical impedance parameter ζ₃ may be defined by the following relationship:

ζ₃=f(Z₃) (Eqn. 6)

where ζ₃ is the statistical impedance parameter for one set 1208, 1218 of cardiac cycles and ƒ(Z₃) is a function of the third impedance vector Z₃. The function ƒ(Z₃) may be a statistical function of the third impedance vector Z₃. For example, the function ƒ(Z₃) may be an average, mean, deviation, standard deviation, maximum, minimum, and the like, of the third impedance vector Z₃. In one specific example, the statistical impedance parameter ζ₃ is the average of the third impedance vector Z₃ in the first set 1208. The statistical impedance parameter ζ₃ is calculated for the first and second groups 1208 and 1218.

The ischemia detection module 274 may calculate a statistical impedance normalization parameter ζ_{N} for each set 1208, 1218 of cardiac cycles. The statistical impedance normalization parameter ζ_{N} may be defined by the following relationship:

ζ_{N}=f(Z_{N}) (Eqn. 7)

where ζ_{N} is the statistical impedance normalization parameter for one set 1208, 1218 of cardiac cycles and ƒ(Z_{N}) is a function of the impedance normalization parameter Z_{N}. The function ƒ(Z_{N}) may be a statistical function of the impedance normalization parameter Z_{N}. For example, the function ƒ(Z_{N}) may be an average, mean, deviation, standard deviation, maximum, minimum, and the like, of the impedance normalization parameter Z_{N}. In one specific example, the statistical impedance normalization parameter ζ_{N} is the average of the impedance normalization parameter Z_{N} measured for each of the cardiac cycles 1200-1206 in the first set 1208. The statistical impedance parameter ζ_{N} is calculated for the first and second sets 1208 and 1218.

The ischemia detection module 274 may calculate a statistical contractility parameter χ₃ for each set 1208, 1218 of cardiac cycles. The statistical contractility parameter χ₃ may be defined by the following relationship:

χ₃=ƒ(C₃) (Eqn. 8)

where χ₃ is the statistical contractility parameter for one set 1208, 1218 of cardiac cycles and ƒ(C₃) is a function of the contractility parameter C₃. The function ƒ(C₃) may be a statistical function of the contractility parameter C₃. For example, the function ƒ(C₃) may be an average, mean, deviation, standard deviation, maximum, minimum, and the like, of the contractility parameter C₃. In one specific example, the statistical contractility parameter C₃ is the average of the contractility parameter C₃ measured for each of the cardiac cycles 1200-1206 in the first set 1208. The statistical contractility parameter C₃ is calculated for the first and second sets 1208 and 1218.

The ischemia detection module 274 may calculate a statistical contractility normalization parameter χ_{N} for each set of cardiac cycles. The statistical contractility normalization parameter χ_{N} may be defined by the following relationship:

χ_{N}=ƒ(C_{N}) (Eqn. 9)

where χ_{N} is the statistical contractility normalization parameter for one set 1208, 1218 of cardiac cycles and ƒ(C_{N}) is a function of the contractility normalization parameter C_{N}. The function ƒ(C_{N}) may be a statistical function of the contractility normalization parameter C_{N}. For example, the function ƒ(C_{N}) may be an average, mean, deviation, standard deviation, maximum, minimum, and the like, of the contractility normalization parameter C_{N}. In one specific example, the statistical contractility normalization parameter χ_{N} is the average of the contractility normalization parameter C_{N} measured for each of the cardiac cycles 1200-1206 in the first set 1208. The statistical contractility parameter χ_{N} is calculated for the first and second sets 1208 and 1218.

The ischemia detection module 274 may calculate a statistical ST segment of interest parameter σ for each set 1208, 1218 of cardiac cycles. The statistical ST segment of interest parameter σ may be defined by the following relationship:

σ=ƒ(SOI) (Eqn. 10)

where σ is the statistical ST segment of interest parameter for one set 1208, 1218 of cardiac cycles and ƒ(SOI) is a function of the segment of interest parameter SOI. The function ƒ(SOI) may be a statistical function of the segment of interest parameter SOI. For example, the function ƒ(SOI) may be an average, mean, deviation, standard deviation, maximum, minimum, and the like, of the segment of interest parameter SOI. In one specific example, the statistical ST segment of interest parameter σ is the average of the segment of interest parameter SOI measured for each of the cardiac cycles 1200-1206 in the first set 1208. The statistical ST segment of interest parameter σ is calculated for the first and second sets 1208 and 1218.

As described above, the ischemia detection module 274 calculates ischemia indicators at 1010 to monitor ischemia in the heart 102 (shown in FIG. 1). The ischemia detection module 274 calculates a group of ischemia indicators for a plurality of sets of cardiac cycles. For example, the ischemia detection module 274 may calculate a group 1224 of ischemia indicators for the first and second sets 1208 and 1218. The group 1224 of ischemia indicators is used to compare the degree of amount of change in the statistical parameters between the first and second statistical parameter groups 1220 and 1222.

The ischemia detection module 274 calculates ischemia indicators from the statistical impedance parameter ζ3, the statistical impedance normalization parameter ζ_{N}, the statistical contractility parameter ζ₃, the statistical contractility normalization parameter χ_{N}, and the statistical ST segment of interest parameter σ for the first and second sets 1208, 1218 of cardiac cycles. For example, the ischemia detection module 274 calculates the amount of change for one or more of the statistical impedance parameter ζ3, the statistical impedance normalization parameter ζ_{N}, the statistical contractility parameter χ₃, the statistical contractility normalization parameter χ_{N}, and the statistical ST segment of interest parameter σ between the first and second sets 1208 and 1218. In one embodiment, the ischemia detection module 274 calculates an impedance ischemia indicator ΔZ₃ for each of a plurality of sets 1208, 1218 of cardiac cycles. For example, the ischemia detection module 274 may calculate the impedance ischemia indicator ΔZ₃ that is the absolute value of the difference between the statistical impedance parameters ζ₃ calculated for the first and second sets 1208 and 1210. The impedance ischemia indicator ΔZ₃ may be represented by the following equation:

$$Δ {Z}_{3} = \vert \frac{{ζ}_{3 ( i )} - {ζ}_{3 ( i + 1 )}}{{ζ}_{3 ( i + 1 )}} \vert \qquad ( Eqn . 11 )$$

where ζ₃₍ᵢ₎ is the statistical impedance parameter ζ₃ for the first set 1208, ζ₃₍ᵢ₊₁₎ is the statistical impedance parameter ζ₃ for the second set 1210, and the impedance ischemia indicator ΔZ₃ is the absolute value of the ratio of the difference between the statistical impedance parameters ζ₃₍ᵢ₎ and ζ₃₍ᵢ₊₁₎ to the statistical impedance parameter ζ₃₍ᵢ₊₁₎.

The ischemia indicators may include an impedance normalization ischemia indicator ΔZ_{N}. The ischemia detection module 274 may calculate the impedance normalization ischemia indicator ΔZ_{N} for each of a plurality of sets of cardiac cycles. For example, the ischemia detection module 274 may calculate the impedance normalization ischemia indicator ΔZ_{N} that is the absolute value of the difference between the statistical impedance normalization parameters ζ_{N} calculated for the first and second sets 1208 and 1210. The impedance normalization ischemia indicator ΔZ_{N} may be represented by the following equation:

$$Δ {Z}_{N} = \vert \frac{{ζ}_{N ( i )} - {ζ}_{N ( i + 1 )}}{{ζ}_{N ( i + 1 )}} \vert \qquad ( Eqn . 12 )$$

where ζ_{N(i)} is the statistical impedance normalization parameter ζ_{N} for the first set 1208, ζ_{N(i+1)} is the statistical impedance normalization parameter ζ_{N} for the second set 1210, and the impedance normalization ischemia indicator ΔZ_{N} is the absolute value of the ratio of the difference between the statistical impedance normalization parameters ζ_{N(i)} and ζ_{N(i+1)} to the statistical impedance parameter ζ_{N(i+1)}.

The ischemia indicators may include a contractility ischemia indicator ΔC₃. The ischemia detection module 274 may calculate the contractility ischemia indicator ΔC₃ for each of a plurality of sets of cardiac cycles. For example, the ischemia detection module 274 may calculate the contractility ischemia indicator ΔC₃ that is the absolute value of the difference between the statistical contractility parameters χ₃ calculated for the first and second sets 1208 and 1210. The contractility ischemia indicator ΔC₃ may be represented by the following equation:

$$Δ {C}_{3} = \vert \frac{{χ}_{3 ( i )} - {χ}_{3 ( i + 1 )}}{{χ}_{3 ( i + 1 )}} \vert \qquad ( Eqn . 13 )$$

where χ₃₍ᵢ₎ is the statistical contractility parameter χ₃ for the first set 1208, χ₃₍ᵢ₊₁₎ is the statistical contractility parameter χ₃ for the second set 1210, and the contractility ischemia indicator ΔC₃ is the absolute value of the ratio of the difference between the statistical contractility parameters χ₃₍ᵢ₎ and χ₃₍ᵢ₊₁₎ to the statistical contractility parameter χ₃₍ᵢ₊₁₎.

The ischemia indicators may include a contractility normalization ischemia indicator ΔC_{N}. The ischemia detection module 274 may calculate the contractility normalization ischemia indicator ΔC_{N} for each of a plurality of sets of cardiac cycles. For example, the ischemia detection module 274 may calculate the contractility normalization ischemia indicator ΔC_{N} that is the absolute value of the difference between the statistical contractility normalization parameters χ_{N} calculated for the first and second sets 1208 and 1210. The contractility normalization ischemia indicator ΔC_{N} may be represented by the following equation:

$$Δ {C}_{N} = \vert \frac{{χ}_{N ( i )} - {χ}_{N ( i + 1 )}}{{χ}_{N ( i + 1 )}} \vert \qquad ( Eqn . 14 )$$

where χ_{N(i)} is the statistical contractility normalization parameter χ_{N} for the first set 1208, χ_{N(i+1)} is the statistical contractility normalization parameter χ_{N} for the second set 1210, and the contractility normalization ischemia indicator ΔC_{N} is the absolute value of the ratio of the difference between the statistical contractility normalization parameters χ_{N(i)} and χ_{N(i+1)} and the statistical contractility normalization parameter χ_{N(i+1)}.

The ischemia indicators may include a segment of interest ischemia indicator ΔSOI. The ischemia detection module 274 may calculate the segment of interest ischemia indicator ΔSOI for each of a plurality of sets of cardiac cycles. For example, the ischemia detection module 274 may calculate the segment of interest ischemia indicator ΔSOI that is the absolute value of the difference between the statistical ST segment of interest parameters σ calculated for the first and second sets 1208 and 1210. The segment of interest ischemia indicator ΔSOI may be represented by the following equation:

$$Δ S O I = \vert \frac{{σ}_{( i )} - {σ}_{( i + 1 )}}{{σ}_{( i + 1 )}} \vert \qquad ( Eqn . 15 )$$

where σ₍ᵢ₎ is the statistical ST segment of interest parameter σ for the first set 1208, σ₍ᵢ₊₁₎ is the statistical ST segment of interest parameter σ for the second set 1210, and the segment of interest ischemia indicator ΔSOI is the absolute value of the ratio of the difference between the statistical ST segment of interest parameters σ₍ᵢ₎ and σ₍ᵢ₊₁₎ to the statistical ST segment of interest parameter σ₍ᵢ₊₁₎.

The ischemia detection module 274 determines how many of the ischemia indicators ΔZ₃, ΔZ_{N}, ΔC₃, ΔC_{N}, and ΔSOI exceed a predetermined minimum threshold at 1012. If a relatively small number of the ischemia indicators do not exceed the threshold, then the ischemia detection module 274 classifies the second cardiac cycle 1218 as non-ischemic. For example, if only one or none of the ischemia indicators exceeds the threshold, then the ischemia detection module 274 classifies the second cardiac cycle 1218 as non-ischemic at 1014. If a relatively large number of the ischemia indicators exceed the threshold, then the ischemia detection module 274 classifies the second cardiac cycle 1218 as ischemic. For example, if four or more of the ischemia indicators exceed the threshold, then the ischemia detection module 274 classifies the second cardiac cycle 1218 as ischemic at 1016. If an intermediate number of the ischemia indicators exceed the threshold, then the ischemia detection module 274 classifies the second cardiac cycle 1218 as potentially ischemic. For example, if two or three of the ischemia indicators exceed the threshold, then the ischemia detection module 274 classifies the second cardiac cycle 1218 as potentially ischemic at 1018. While the above examples compare the ischemia indicators to a single threshold, multiple thresholds may be used in another embodiment. Moreover, the number of ischemia indicators that must exceed the threshold before classifying the second set 1218 as ischemic, potentially ischemic or non-ischemic may differ from those described above.

If the ischemia detection module 274 classifies the second set 1218 of cardiac cycles 1210-1216 as potentially ischemic at 1018, then the ischemia detection module 274 may perform a secondary check on whether the heart 102 (shown in FIG. 1) is ischemic. For example, the ischemia detection module 274 may calculate a sum Σ of two or more of the ischemia indicators at 1020 and compare this sum Σ is a predetermined minimum sum, or threshold, at 1022. The sum Σ may be represented as follows:

Σ=ΔZ₃+ΔZ_{N}+ΔC₃+ΔC_{N}+ΔSOI (Eqn. 16)

If the sum Σ exceeds the predetermined minimum sum, then the ischemia detection module 274 classifies the second set 1218 of cardiac cycle 1210-1216 as ischemic at 1016. If the sum Σ does not exceed the minimum sum, then the ischemia detection module 274 classifies the second set 1218 of cardiac cycles 1210-1216 as 1014. By way of nonlimiting example only, the predetermined minimum sum is 10%. Other minimum sums may be used, such as 8%, 6%, 12%, 14%, and the like. The predetermined minimum sum may be stored at the memory 244 (shown in FIG. 3).

Alternatively, the ischemia detection module 274 compares the sum Σ to a plurality of predetermined minimum sums. The ischemia detection module 274 may compare the sum Σ to a lower predetermined minimum sum and an upper predetermined minimum sum. By way of non-limiting example only, the ischemia detection module 274 may compare the sum Σ to a lower minimum sum of 5% and to an upper minimum sum of 10%. If the sum Σ does not exceed the lower minimum sum, the ischemia detection module 274 classifies the current cardiac cycles as non-ischemic. If the sum Σ exceeds the lower minimum sum but does not exceed the upper minimum sum, the ischemia detection module 274 classifies the second set 1218 of cardiac cycles 1210-1216 as potentially ischemic. If the sum Σ exceeds the upper minimum sum, the ischemia detection module 274 classifies the second set 1218 of cardiac cycles 1210-1216 as ischemic.

In another embodiment, the ischemia detection module 274 detects ischemia in the heart 102 (shown in FIG. 1) when the ischemia detection module 274 classifies a plurality of sets 1208, 1218 of cardiac cycles as ischemic. The ischemia detection module 274 may notify an operator of the IMD 100 that ischemia is detected when a predetermined minimum number of sets 1208, 1218 of cardiac cycles are classified as ischemic according to one or more of the embodiments described above. For example, the ischemia detection module 274 may determine that ischemia is detected when at least a minimum number of consecutive sets 1208, 1218 of cardiac cycles are classified as being ischemic, as described above. Alternatively, the ischemia detection module 274 may notify an operator of the IMD 100 that ischemia is detected when consecutive sets 1208, 1218 of cardiac cycles are classified as ischemic for a minimum amount of time according to one or more of the embodiments described above. The predetermined minimum time may be stored in the memory 244 (as shown in FIG. 3). For example, the ischemia detection module 274 may determine that ischemia is detected when the cardiac cycles over a previous time period are classified as ischemic according to one or more embodiments described above.

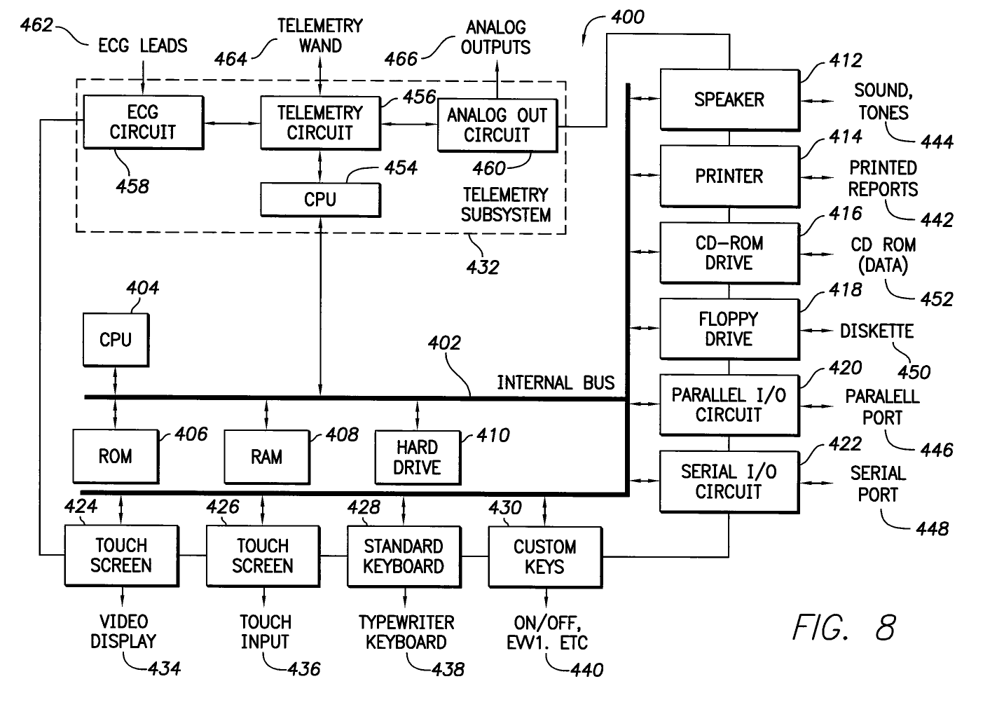

The ischemia detection module 274 may communicate the classification of the second set 1218 of cardiac cycles 1210-1216 as ischemic, potentially ischemic or non-ischemic to an operator of the IMD 100. For example, the classification of the second set 1218 of cardiac cycles 1210-1216 may be visually communicated on a display 424 (shown in FIG. 8). The process 1000 may continue to monitor the heart 102 for ischemia after the second set 1218 of cardiac cycles 1210-1216 is classified by the ischemia detection module 274 as ischemic, non-ischemic or potentially ischemic. For example, the process 1000 may continue in a loop-wise manner as shown in FIG. 4.

FIG. 6 illustrates an alternative manner of measuring the third impedance vector Z₃ according to one embodiment. The positions of first, second and third impedance vectors Z₁, Z₂ and Z₃ shown in FIG. 6 are approximate and are provided as illustrations. The actual positions of the first, second and third impedance vectors Z₁, Z₂ and Z₃ may slightly differ from the positions shown in FIG. 6. Similar to the embodiment shown in FIG. 1, the first impedance vector Z₁ extends between the housing 104 and the RV coil electrode 134 and the second impedance vector Z₂ extends between the housing 104 and the SVC coil electrode 138. The current I₃ is supplied between the SVC coil electrode 138 and the LV ring electrode 130. The current I₃ is supplied by electrically connecting the SVC coil electrode 138 and the LV ring electrode 130 to a source of electric current, such as the battery 256 (shown in FIG. 3). Similar to the embodiment shown in FIG. 1, the voltage V₃ is measured between the LV electrode tip 124 and the RV electrode tip 136 in one embodiment. The voltage V₃ includes the voltage difference between the LV and RV electrode tips 124 and 136 measured from the current I₃. The third impedance vector Z₃ can be calculated as described above. The SVC coil electrode 138 may have a lower intrinsic impedance than other electrodes in the IMD 100. For example, the SVC coil electrode 138 may have an intrinsic impedance of approximately 100 ohms or less. Using electrodes with lower intrinsic impedances to supply the current I₃, measure the voltage V₃, the first impedance vector Z₁ and/or the second impedance vector Z₂ can increase the sensitivity of the IMD 100 to smaller changes in the impedance of the myocardium of the heart 102.

The current I₃ may be applied at one or more of a variety of frequencies in one or more of the embodiments described herein. One or more frequencies at which the current I₃ is applied may cause the IMD 100 to be more sensitive to smaller changes in one or more of the impedance vectors Z₁, Z₂ and Z₃. For example, the IMD 100 may measure a relatively small change in the third impedance vector Z₃ when the current I₃ is applied at a first frequency but not measure the same change in the third impedance vector Z₃ when the current I₃ is applied at a second, different frequency.

FIG. 8 illustrates a functional block diagram of the external device 400, such as a programmer, that is operated by a physician, a health care worker, or a patient to interface with IMD 100 (shown in FIG. 1). The external device 400 may be utilized in a hospital setting, a physician's office, or even the patient's home to communicate with the IMD 100 to change a variety of operational parameters regarding the therapy provided by the IMD 100 as well as to select among physiological parameters to be monitored and recorded by the IMD 100. For example, the external device 400 may be used to program coronary episode related parameters, such as ischemia-related and AMI-related ST segment shift thresholds, duration thresholds, and the like. Further, the external device 400 may be utilized to interrogate the IMD 100 to determine the condition of a patient, to adjust the physiological parameters monitored or to adapt the therapy to a more efficacious one in a non-invasive manner.

External device 400 includes an internal bus 402 that connects/interfaces with a Central Processing Unit (CPU) 404, ROM 406, RAM 408, a hard drive 410, a speaker 412, a printer 414, a CD-ROM drive 416, a floppy drive 418, a parallel I/O circuit 420, a serial I/O circuit 422, the display 424, a touch screen 426, a standard keyboard connection 428, custom keys 430, and a telemetry subsystem 432. The internal bus 402 is an address/data bus that transfers information (e.g., either memory data or a memory address from which data will be either stored or retrieved) between the various components described. The hard drive 410 may store operational programs as well as data, such as reference ST segments, ST thresholds, impedance thresholds, other thresholds, timing information and the like.

The CPU 404 typically includes a microprocessor, a micro-controller, or equivalent control circuitry, designed specifically to control interfacing with the external device 400 and with the IMD 100 (shown in FIG. 1). The CPU 404 may further include RAM or ROM memory, logic and timing circuitry, state machine circuitry, and I/O circuitry to interface with the IMD 100. Typically, the microcontroller 222 (shown in FIG. 2) includes the ability to process or monitor input signals (e.g., data) as controlled by program code stored in memory (e.g., ROM 406).

The display 424 (e.g., may be connected to a video display 434) and the touch screen 426 display text, alphanumeric information, data and graphic information via a series of menu choices to be selected by the user relating to the IMD 100, such as for example, status information, operating parameters, therapy parameters, patient status, access settings, software programming version, ST segment thresholds, impedance thresholds, other thresholds, and the like. The touch screen 426 accepts a user's touch input 436 when selections are made. The keyboard 428 (e.g., a typewriter keyboard 438) allows the user to enter data to the displayed fields, operational parameters, therapy parameters, as well as interface with the telemetry subsystem 432. Furthermore, custom keys 430 turn on/off 440 (e.g., EVVI) the external device 400. The printer 414 prints hard-copies of reports 442 for a physician/healthcare worker to review or to be placed in a patient file, and speaker 412 provides an audible warning (e.g., sounds and tones 444) to the user in the event a patient has any abnormal physiological condition occur while the external device 400 is being used. The parallel I/O circuit 420 interfaces with a parallel port 446. The serial I/O circuit 422 interfaces with a serial port 448. The floppy drive 418 accepts diskettes 450. The CD-ROM drive 416 accepts CD ROMs 452.

The telemetry subsystem 432 includes a central processing unit (CPU) 454 in electrical communication with a telemetry circuit 456, which communicates with both an ECG circuit 458 and an analog out circuit 460. The ECG circuit 458 is connected to ECG leads 462. The telemetry circuit 456 is connected to a telemetry wand 464. The analog out circuit 432 includes communication circuits, such as a transmitting antenna, modulation and demodulation stages (not shown), as well as transmitting and receiving stages (not shown) to communicate with analog outputs 466. The external device 400 may wirelessly communicate with the IMD 100 and utilize protocols, such as Bluetooth, GSM, infrared wireless LANs, HIPERLAN, 3G, satellite, as well as circuit and packet data protocols, and the like. A wireless RF link utilizes a carrier signal that is selected to be safe for physiologic transmission through a human being and is below the frequencies associated with wireless radio frequency transmission. Alternatively, a hard-wired connection may be used to connect the external device 400 to IMD 100 (e.g., an electrical cable having a USB connection).

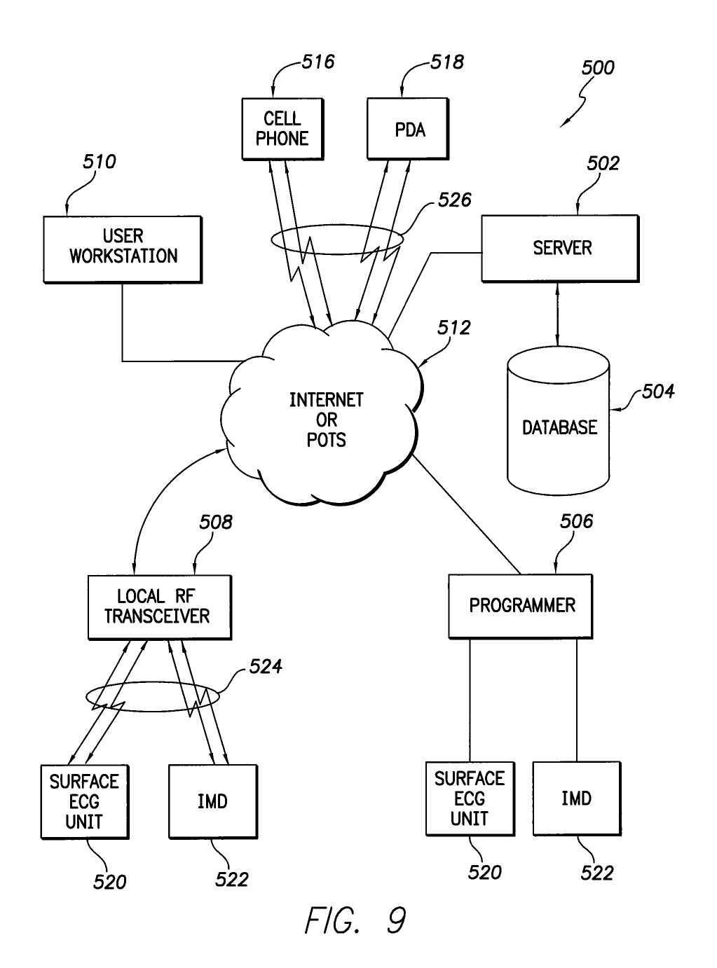

FIG. 9 illustrates a distributed processing system 500 in accordance with one embodiment. The distributed processing system 500 includes a server 502 that is connected to a database 504, a programmer 506 (e.g., similar to external device 400 described above and shown in FIG. 8), a local RF transceiver 508 and a user workstation 510 electrically connected to a communication system 512. The communication system 512 may be the internet, a voice over IP (VoIP) gateway, a local plain old telephone service (POTS) such as a public switched telephone network (PSTN), and the like. Alternatively, the communication system 512 may be a local area network (LAN), a campus area network (CAN), a metropolitan area network (MAN), or a wide area network (WAM). The communication system 512 serves to provide a network that facilitates the transfer/receipt of cardiac signals, processed cardiac signals, histograms, trend analysis and patient status, and the like.

The server 502 is a computer system that provides services to other computing systems (e.g., clients) over a computer network. The server 502 acts to control the transmission and reception of information (e.g., cardiac signals, processed cardiac signals, ST segments, R-waves, thresholds, impedances, histograms, statistical analysis, trend lines, and the like). The server 502 interfaces with the communication system 512, such as the internet or a local POTS based telephone system, to transfer information between the programmer 506, the local RF transceiver 508, the user workstation 510 as well as a cell phone 516, and a personal data assistant (PDA) 518 to the database 504 for storage/retrieval of records of information. For instance, the server 502 may download, via a wireless connection 526, to the cell phone 516 or the PDA 518 the results of processed cardiac signals, ST segment trends, impedance vectors, or a patient's physiological state (e.g., is the patient having or has had an ischemia) based on previously recorded cardiac information. On the other hand, the server 502 may upload raw cardiac signals (e.g., unprocessed cardiac data) from a surface ECG unit 520 or an IMD 522 via the local RF transceiver 508 or the programmer 506.

Database 504 is any commercially available database that stores information in a record format in electronic memory. The database 504 stores information such as raw cardiac data, processed cardiac signals, statistical calculations (e.g., averages, modes, standard deviations), histograms, cardiac trends (e.g., STS trends), and the like. The information is downloaded into the database 504 via the server 502 or, alternatively, the information is uploaded to the server from the database 504.

The programmer 506 is similar to the external device 400 shown in FIG. 6 and described above, and may reside in a patient's home, a hospital, or a physician's office. Programmer 506 interfaces with the surface ECG unit 520 and the IMD 522 (e.g., similar to the IMD 100 described above and shown in FIG. 1). The programmer 506 may wirelessly communicate with the IMD 522 and utilize protocols, such as Bluetooth, GSM, infrared wireless LANs, HIPERLAN, 3G, satellite, as well as circuit and packet data protocols, and the like. Alternatively, a hard-wired connection may be used to connect the programmer 506 to IMD 100 (e.g., an electrical cable having a USB connection). The programmer 506 is able to acquire cardiac signals from the surface of a person (e.g., ECGs), or the programmer is able to acquire intra-cardiac electrogram (e.g., IEGM) signals from the IMD 522. The programmer 506 interfaces with the communication system 512, either via the internet or via POTS, to upload the cardiac data acquired from the surface ECG unit 520 or the IMD 522 to the server 502. The programmer 506 may upload more than just raw cardiac data. For instance, the programmer 506 may upload status information, operating parameters, therapy parameters, patient status, access settings, software programming version, ST segment thresholds, calculated or measured impedance vectors, and the like.

The local RF transceiver 508 interfaces with the communication system 512, either via the internet or via POTS, to upload cardiac data acquired from the surface ECG unit 520 or the IMD 522 to the server 502. In one embodiment, the surface ECG unit 520 and the IMD 522 have a bi-directional connection with the local RF transceiver via a wireless connection. The local RF transceiver 508 is able to acquire cardiac signals from the surface of a person (e.g., ECGs), or acquire intra-cardiac electrogram (e.g., IEGM) signals from the IMD 522. On the other hand, the local RF transceiver 508 may download stored cardiac data from the database 504 or the analysis of cardiac signals from the database 504 (e.g., ST segment statistical analysis, ST segment trends, impedance vectors, and the like) information to the surface ECG unit 520 or the IMD 522.

The user workstation 510 may interface with the communication system 512 via the internet or POTS to download information via the server 502 from the database 504. Alternatively, the user workstation 510 may download raw data from the surface ECG unit 520 or IMD 522 via either the programmer 506 or the local RF transceiver 508. Once the user workstation 510 has downloaded the cardiac information (e.g., raw cardiac signals, ST segments, impedance vectors, and the like), the user workstation 510 may process the cardiac signals, create histograms, calculate statistical parameters, or determine cardiac trends and determine if the patient is suffering from ischemia or another physiological condition. Once the user workstation 510 has finished performing its calculations, the user workstation 510 may either download the results to the cell phone 516, the PDA 518, the local RF transceiver 508, the programmer 506, or to the server 502 to be stored on the database 504.

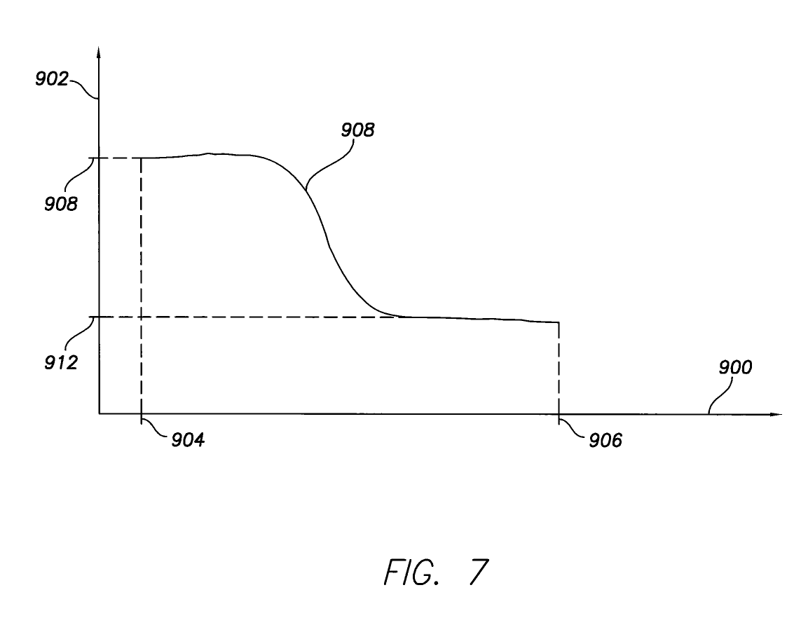

FIG. 7 is an illustration of the third impedance vector Z₃ over a range of frequencies of the current I₃ according to one embodiment. The horizontal axis 900 represents the frequency of the current I₃ supplied to the electrodes according to one or more of the embodiments described herein. The vertical axis 902 represents the third impedance vector Z₃ determined according to one or more of the embodiments described herein. As described above, an operator may sweep the frequency of the current I₃ from a lower frequency 904 to a higher frequency 906. At frequencies near the lower frequency 904, the third impedance vector Z₃ remains approximately constant at an upper impedance 908. For example, at frequencies proximate to 500 Hz, the third impedance vector Z₃ may be approximately 100 ohms. As the frequency of the current I₃ is increased to the higher frequency 906, the third impedance vector Z₃ may gradually decrease as shown by the curve 910. At frequencies that approach the higher frequency 906, the third impedance vector Z₃ becomes approximately constant to a lower impedance 912. For example, at frequencies proximate to 10 kHz, the third impedance vector Z₃ may reduce to approximately 50 ohms.

An operator can determine a frequency of the current I₃ that is more sensitive to changes in the third impedance vector Z₃ brought on by ischemia by examining the sensitivity or degree of change in the third impedance vector Z₃ over a range of frequencies before and after ischemia is induced in the patient. For example, the operator can sweep the frequencies at which the current I₃ is applied between a lower frequency 904 of 100 Hz and a higher frequency 906 of 50 kHz. The frequency may be swept through this frequency range once or repeated times over a 10 to 30 second time interval. The curve 908 defining the third impedance vector Z₃ over the frequency range may be displayed to the operator and/or stored in the memory 244 (shown in FIG. 3). Ventricular fibrillation may then be induced in the patient by, for example, inflating an angioplasty balloon in the patient. As ventricular fibrillation is induced, the heart 102 can become more ischemic. The frequency of the current I₃ is then swept between the lower and higher frequencies 904 and 906, and another curve 908 that defines the third impedance vector Z₃ over this frequency range is obtained. These two curves 908 may be compared to determine the frequency or frequencies at which the current I₃ is applied that are more sensitive to changes in the third impedance vector Z₃ brought about by ischemia. For example, the previous curve 908 that is obtained prior to inducing ischemia may be used as a baseline to compare the later obtained curve 908. Variations between the previous and later obtained curves 908 at one or more frequencies can reveal the frequencies at which the IMD 100 is more sensitive to changes in the impedance of the heart 102 brought about by ischemia.

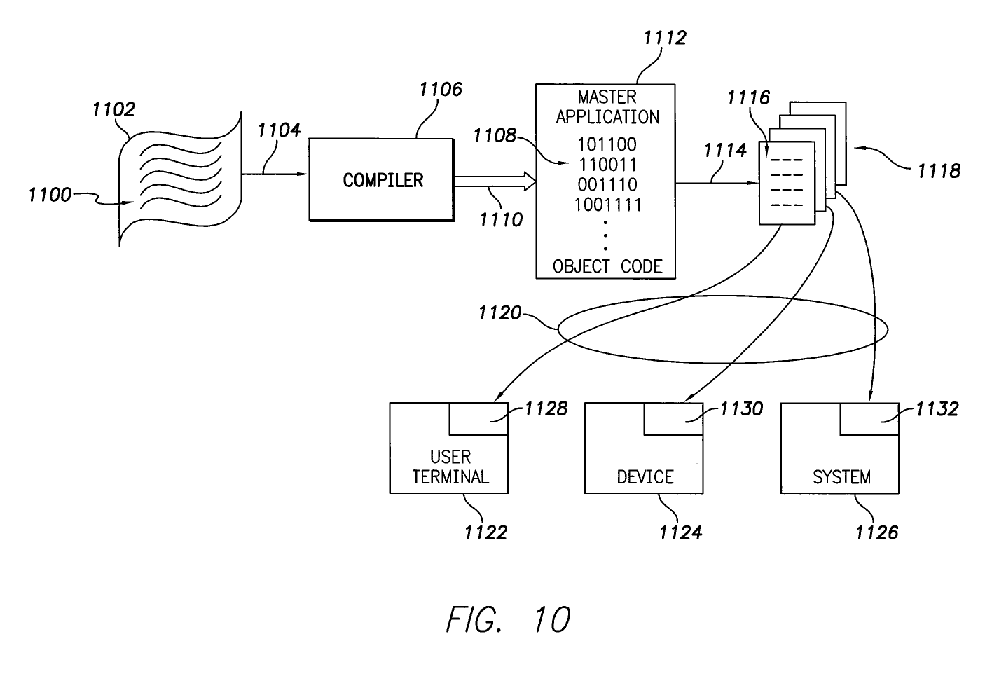

FIG. 10 illustrates a block diagram of exemplary manners in which embodiments of the present invention may be stored, distributed and installed on a computer-readable medium. In FIG. 10, the “application” represents one or more of the methods and process operations discussed above. For example, the application may represent the process carried out in connection with FIGS. 1 through 9 as discussed above.

As shown in FIG. 10, the application is initially generated and stored as source code 1100 on a source computer-readable medium 1102. The source code 1100 is then conveyed over path 1104 and processed by a compiler 1106 to produce object code 1108. The object code 1108 is conveyed over path 1110 and saved as one or more application masters on a master computer-readable medium 1112. The object code 1108 is then copied numerous times, as denoted by path 1114, to produce production application copies 1116 that are saved on separate production computer-readable medium 1118. The production computer-readable medium 1118 is then conveyed, as denoted by path 1120, to various systems, devices, terminals and the like. In the example of FIG. 10, a user terminal 1122, a device 1124 and a system 1126 are shown as examples of hardware components, on which the production computer-readable medium 1118 are installed as applications (as denoted by 1128 through 1132). For example, the production computer-readable medium 1118 may be installed on the IMD 100 (shown in FIG. 1) and/or the controller 400 (shown in FIG. 8).

The source code may be written as scripts, or in any high-level or low-level language. Examples of the source, master, and production computer-readable medium 1102, 1112 and 1118 include, but are not limited to, CDROM, RAM, ROM, Flash memory, RAID drives, memory on a computer system and the like. Examples of the paths 1104, 1110, 1114, and 1120 include, but are not limited to, network paths, the internet, Bluetooth, GSM, infrared wireless LANs, HIPERLAN, 3G, satellite, and the like. The paths 1104, 1110, 1114, and 1120 may also represent public or private carrier services that transport one or more physical copies of the source, master, or production computer-readable medium 1102, 1112 or 1118 between two geographic locations. The paths 1104, 1110, 1114 and 1120 may represent threads carried out by one or more processors in parallel. For example, one computer may hold the source code 1100, compiler 1106 and object code 1108. Multiple computers may operate in parallel to produce the production application copies 1116. The paths 1104, 1110, 1114, and 1120 may be intra-state, inter-state, intra-country, inter-country, intra-continental, inter-continental and the like.

The operations noted in FIG. 10 may be performed in a widely distributed manner world-wide with only a portion thereof being performed in the United States. For example, the application source code 1100 may be written in the United States and saved on a source computer-readable medium 1102 in the United States, but transported to another country (corresponding to path 1104) before compiling, copying and installation. Alternatively, the application source code 1100 may be written in or outside of the United States, compiled at a compiler 1106 located in the United States and saved on a master computer-readable medium 1112 in the United States, but the object code 1108 transported to another country (corresponding to path 1114) before copying and installation. Alternatively, the application source code 1100 and object code 1108 may be produced in or outside of the United States, but production application copies 1116 produced in or conveyed to the United States (for example, as part of a staging operation) before the production application copies 1116 are installed on user terminals 1122, devices 1124, and/or systems 1126 located in or outside the United States as applications 1128 through 1132.

As used throughout the specification and claims, the phrases “computer-readable medium” and “instructions configured to” shall refer to any one or all of (i) the source computer-readable medium 1102 and source code 1100, (ii) the master computer-readable medium and object code 1108, (iii) the production computer-readable medium 1118 and production application copies 1116 and/or (iv) the applications 1128 through 1132 saved in memory in the terminal 1122, device 1124 and system 1126.

In accordance with certain embodiments, methods and systems are provided that are able to monitor ischemia using variations in one or more segment of interest and variations in one or more impedance vectors. The use of both segment and impedance variations can improve the accuracy of detecting ischemia in a patient.

It is to be understood that the above description is intended to be illustrative, and not restrictive. For example, the above-described embodiments (and/or aspects thereof) may be used in combination with each other. In addition, many modifications may be made to adapt a particular situation or material to the teachings of the invention without departing from its scope. While the dimensions and types of materials described herein are intended to define the parameters of the invention, they are by no means limiting and are exemplary embodiments. Many other embodiments will be apparent to those of skill in the art upon reviewing the above description. The scope of the invention should, therefore, be determined with reference to the appended claims, along with the full scope of equivalents to which such claims are entitled. In the appended claims, the terms “including” and “in which” are used as the plain-English equivalents of the respective terms “comprising” and “wherein.” Moreover, in the following claims, the terms “first,” “second,” and “third,” etc. are used merely as labels, and are not intended to impose numerical requirements on their objects. Further, the limitations of the following claims are not written in means-plus-function format and are not intended to be interpreted based on 35 U.S.C. § 112, sixth paragraph, unless and until such claim limitations expressly use the phrase “means for” followed by a statement of function void of further structure.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. An implantable medical device, comprising:
at least one leads comprising electrodes configured to be positioned within a heart, the electrodes being capable of sensing cardiac signals having a segment of interest;
a segment monitoring module to determine segment variations of the segment of interest in the cardiac signals;
an impedance detection module to measure impedance vectors between predetermined combinations of the electrodes; and
an ischemia detection module to monitor ischemia based on changes in the segment variations of the segment of interest and based on changes in the impedance vectors.

2. The device of claim 1, wherein the ischemia detection module derives a set of parameter changes based on the impedance vectors measured, and the segment variations determined, between at least one current and at least one prior cardiac cycle, the parameter changes being used to monitor ischemia.

3. The device of claim 2, wherein the ischemia detection module determines how many of the parameter changes exceed a threshold, and classifies at least one of the cardiac cycles as one of ischemic, non-ischemic and potentially ischemic based on how many of the parameters changes exceed the threshold.

4. The device of claim 2, wherein the ischemia detection module sums a plurality of the parameter changes, determines whether a sum of the summed parameter changes exceeds a threshold, and classifies at least one of the cardiac cycles as one of ischemic, non-ischemic and potentially ischemic based on whether the sum exceeds the threshold.

5. The device of claim 1, wherein the ischemia detection module calculates a relative change in impedance between a current set of impedance vectors and a prior set of impedance vectors and based thereon, monitors ischemia.

6. The device of claim 1, wherein the ischemia detection module calculates impedance parameters and contractility parameters based on the measured impedance vectors to monitor ischemia based thereon.

7. The device of claim 1, wherein the segment monitoring module determines ST segment variations over multiple cardiac cycles, and based thereon calculates a statistical ST segment parameter, the statistical ST segment parameter constituting at least one of mean, median, average, deviation, maximum, and minimum ST segment variation over the multiple cardiac cycles, the ischemia detection module using the statistical ST segment parameter to monitor ischemia.

8. The device of claim 1, wherein the impedance detection module obtains first and second impedance vectors along first and second paths, and calculates a normalized impedance parameter based on a ratio of the first and second impedance vectors to at least partially correct for changes in the first and second impedance vectors that are due to physiologic characteristics unrelated to ischemia.

9. The device of claim 1, wherein the impedance detection module obtains a first impedance vector along a path primarily traversing the heart and obtains a second impedance vector along a path traversing at least a portion of a lung.

10. The device of claim 1, wherein the impedance vectors represent impedance values measured between corresponding combinations of the electrodes.

11. The device of claim 1, wherein the segment of interest represents an ST segment.

12. The device of claim 1, wherein at least one of the leads includes at least one pacing electrode to deliver pacing stimulus, the impedance detection module measuring at least one impedance vector utilizing the pacing electrode.

13. The device of claim 1, wherein the electrodes include defibrillation electrodes and pacing electrodes, the device further comprising a current source to deliver a current between the defibrillation electrodes, the impedance detection module measuring at least one impedance vector between the pacing electrodes.

14. The device of claim 1, wherein the electrodes include defibrillation electrodes and pacing electrodes, the impedance detection module measuring a first impedance vector between a pair of defibrillation electrodes, the impedance detection module measuring a second impedance vector between one of the pacing electrodes and one of the defibrillation electrodes.

15. The device of claim 1, wherein at least one of the electrodes, utilized to measure the impedance vectors, has an intrinsic impedance of at least 500 ohms.

16. The device of claim 1, wherein the electrodes utilized to measure the impedance vectors include at least one of a RV and LV tip electrode and include at least one of an RV coil, LV ring, and SVC coil electrode having an intrinsic impedance of less than 100 ohms.

17. A method for monitoring ischemia, comprising:
providing leads that include electrodes that are configured to be positioned within a heart;
sensing, with the electrodes, cardiac signals having a segment of interest;
determining segment variations of the segment of interest in the cardiac signals;
measuring impedance vectors between predetermined combinations of the electrodes; and
monitoring ischemia based on changes in the segment variations of the segment of interest and based on changes in the impedance vectors.

18. The method of claim 17, further comprising deriving a set of parameter changes based on the impedance vectors and the segment variations between at least one current and at least one prior cardiac cycle.

19. The method of claim 17, further comprising calculating impedance parameters and contractility parameters based on the measured impedance vectors to monitor ischemia.

20. The method of claim 17, wherein the monitoring comprises calculating a relative change in impedance between a current set of impedance vectors and a prior set of impedance vectors and based thereon, monitoring ischemia.
